(19) United States
(12) Patent Application Publication
(10) Pub. No.: US 2008/0255174 A1
(43) Pub. Date: Oct. 16, 2008

(54) Hydroalcoholic Extract Of Erythrina Mulungu, Pharmaceutical Compositions And Processes For Producing These Substances

(76) Inventors: Bolzani Vanderlan da silva, Araraquara-SP (BR); Ricardo Luis Nunes de Souza, Araraquara-SP (BR); Flausino Otavio Aparecido, Araraquara-SP (BR)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 11/576,895

(22) PCT Filed: Oct. 20, 2005
(86) PCT No.: PCT/BR2005/000217

§ 371 Date: Feb. 15, 2008

(30) Foreign Application Priority Data
Oct. 20, 2004 (BR) PI 040.6124-1

(51) Int. Cl. A61K 31/438; C07D 471/10

(52) U.S. Cl. 514/278; 546/18

(57) ABSTRACT

The use of molecules for cholinergic and/or serotonergic system models, revealing pharmaceutical compositions comprising 11-OH-erythravine, erythravine, erythrartine, pharmaceutically acceptable isotherals, salts, byproducts and/or solvates thereof, optionally containing other erythrina byproducts, for the treatment of anxiety disorders; processes for obtaining said pharmaceutical compositions are also revealed.

### FIELD OF INVENTION

The present invention refers to molecules acting on the cholinergic and/or serotonergic systems. More specifically, the present invention refers to erythrina byproducts useful in the preparation of anxiolytic medicines. Pharmaceutical compositions comprising said molecules and processes for preparing said pharmaceutical compositions are also provided.

### BACKGROUND TO THE INVENTION

Erythrina mulungu (Papilionaceae-Leguminoseae) is an arboreal plant (10-14 meters high) having red florescence, and grows in the semideciduous latifoliate forests of the Paraná basin and in scrubland regions, principally the western region of the State of São Paulo and Minas Triangle (LORENZI, 1992). The plant's bark is used by the local population as a tranquilizer and sedative. It is popularly known as mulungu, coral tree, coral mulungu, coral shrub (ethnic names include capa-homem, suiná-suiná, tiriceiro among others) (LORENZI, 1992). Eight varieties of Erythrina are found in Brazil: E. mulungu, E. velutina, E. crita-galli, E. poeppigiana, E. fusca, E. falcata, E. speciosa and E. verna (LORENZI, 1992). Despite the scarcity of studies of the E. mulungu species, much work has been carried out to ascertain the fitochemical and pharmacological properties of other species of the variety, which are also known for their popular use as a sedative, tranquilizer and also as a laxative, anti-inflammatory and anti-diuretic agent (GARÍN-AGUILAR et al. 2000).

### Fitochemistry

Interest in the study of the Erythrina species began in 1877 when Domínguez and Altamirano discovered that the pharmacological action of the seed extract of E. americana was similar to the effects of d-tubocurarine (substance extracted from Chondodendron tomentosum) (HARGREAVES et al. 1974; HIDER et al. 1986; GARÍN-AGUILAR et al, 2000). From that point in time, investigations were carried out on the fitochemical and pharmacological properties of the extracts of different species of Erythrina. Years later, after confirmation of the pharmacological action displayed in the extracts of various species of Erythrina, research intensified towards isolating and identifying the alkaloids in plants of this kind (SARRAGIOTO et al., 1981). Up until such time, pharmacological testing was performed on crude extracts. In 1937, Folkers and Major (1937) performed chemical investigations on the seeds of E. americana Mill. and isolated a crystalline alkaloid, erythroidine, which presented cholinergic activity similar to that of d-tubocurarine. Subsequent analyses (BOEKELHEIDE and GRUNDON, 1953; BOEKELHEIDE et al., 1953) showed that erythroidine was a mixture of two isomeric alkaloids denominated α-erythroidine and β-erythroidine, the latter being responsible for the cholinergic activity (HARGREAVES et al., 1974; HIDER et al., 1986; GARÍN-AGUILAR, at al., 2000). After isolating α and β-erythroidine of E. americana, other species of Erythrina were studied, resulting in the isolation of new erythrina skeleton alkaloids (FOLKERS and KONIUSZY, 1940; FOLKERS et al., 1944; BOEKELHEIDE and GRUNDON, 1953; BOEKELHEIDE et al., 1953; TANDON et al., 1969; ITO et al. 1970; BARTON et al., 1970; GHOSAL, 1970; GHOSAL et al., 1971; ITO et at, 1971; MIANA et al., 1972; GHOSAL et al., 1972 a,b; BARTON et al., 1973; ITO et al., 1973, a,b,c,d; GHOSAL and SRIVASTAVA, 1974; MILLINGTON et al., 1974; GAMES et al., 1974; ITO et al., 1976; BARAKAT et al., 1977; EL-OLEMY et al., 1978; AHMAD et al., 1979; TIVVARI and MASSOD, 1979a,b; SARRAGIOTO et al., 1981).

Clarification of the basic structure of the erythrina alkaloids was achieved by degradation and synthesis (GRUNDON and BOEKELHEIDE, 1953; GRUNDON et al., 1953; WEINSTOCK and BOEKELHEIDE, 1953; BOEKELHEIDE et al., 1953). The presence of a spiroaminic skeleton was established in the structure of these alkaloids, which present the general formula below (representing an erythrina alkaloid of the dienic type).

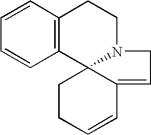

Clarification of this structure facilitated the subsequent identification of the new compounds. Currently, three types of erythrina alkaloids are known. The dienoids present a dienic system in rings A and B. The alkaloids have a double bond Δ^{1,6} in ring A. A third group of erythrina alkaloids includes: erysodienone, 3-desmethoxy erythratidinone, α-erythroidine and β-erythroidine. Some alkaloids of certain Erythrina species not presenting the erythrina skeleton were also isolated, including: orientaline, N-Noorientaline, protosinomenine, N-Norprotosinomenine, isoboldine, erybidine, scoureline, coreximine, hypaforin, coline.

A fitochemical study of E. mulungu using ethanol extract prepared with the dried flowers isolated five alkaloids (erysothrina, N-erysothrina oxide, erythrartine, N-erythrartine oxide and hypaforin) and a terpenoid, fithol (SARRAGIOTO et al., 1981; SARRAGIOTO, 1981). Recent fitochemical studies have demonstrated that species of Erythrina are also rich in other classes of substances, such as flavanones, isoflavanones, isoflavones and pterocarpanes (DA-CUNHA et al. 1998; TANAKA et al., 1996, 1997a,b; 1998; 2001; OH et al. 1999; YENESEW et al. 2000; NKENGFACK et al., 2001).

### Pharmacological Activities

Among the principal pharmacological actions of substances obtained from Erythrina is its peripherical activity on the cholinergic system, which has been compared to the effects of d-tubocurarine (HARGREAVES et al., 1974; HIDER et al. 1986; GARÍN-AGUILAR, et al. 2000). This effect was attributed to the alkaloid dihydro-β-erythroidine (DHBE), a nicotine-like antagonist receptor (HIDER et al., 1986) isolated from E. americana (BOEKELHEIDE and GRUNDON, 1953; BOEKELHEIDE et al., 1953) and E. tholloniana (CHAVVLA et al, 1985). More recently, in an in vitro test, DHBE was characterized as a serotonergic 3 antagonist receptor (5-HT₃) (ELSELÈ et al., 1993). Some species of the Erythrina variety also present other activities on the Central Nervous System, such as an anticonvulsant, hypnotic, anesthetic, sedative and anxiolytic effect (GHOSAL et al. 1972; HARGREAVES et al., 1974; RATNASOORIYA and DHARMASIRI, 1999; ONUSIC et al., 2002). However, no reported study has analyzed the role of erythrina alkaloids in these activities.

A study into E. velutina demonstrated that acute treatment with hydroalcoholic extract decreased the activity of the mice in the open-field test (with doses of 250 and 500 mg/kg, oral intake), and also increased the period of sleep induced by pentobarbital and the period for the start of pilocarpine-induced convulsion (with doses of 500 and 1000 mg/kg, oral intake), indicating a depressive effect on the Central Nervous System (CABRAL et al., 2000). Another work (GARÍN-AGULIAR et al. 2000) revealed that acute treatment with the hexanic fraction of E. americana (3 mg/kg, i.p) decreased the aggressive behavior in male mice, similarly to diazepam. Recently, a study on the hydroalcoholic extract of E. mulungu (ONUSIC et al., 2002) observed that acute treatment with a dose of 200 mg/kg (oral intake) presented an anxiolytic effect on mice in the inhibitory avoidance task in the elevated T maze test, comparable to that of the benzodiazepinic anxiolytic (BDZ), diazepam. ONUSIC and collaborators (2002) also observed the anxiolytic effect of E. mulungu, with the same dose, in the light/dark transition model, both in the number of transitions between the two model compartments as in the length of stay in the light compartment. Another work revealed that chronic treatment oral intake (9 days) with the extract of E. mulungu presented an anxiolytic effect, with doses of 50, 100 and 200 mg/kg, both in the inhibitory avoidance task, as in the escape from the open arms of the elevated T maze (ONUSIC et al., 2003). In the light/dark transition model, the extract of E. mulungu, with a dose of 50 mg/kg, also presented an anxiolytic effect after chronic treatment for 14 days (ONUSIC et al. 2003).

Despite these innumerous approaches, to-date there is no known report of the development of anxiolytic medicines made from isolated active principles of Erythrina, or from the chemical synthesis thereof, nor any processes for preparing such medicines.

### SUMMARY OF THE INVENTION

The subject matter of the present invention is to provide molecules capable of acting on the cholinergic and/or serotonergic systems and pharmaceutical compositions comprising same.

In one aspect, the molecules of the present invention proved to be active anxiolytics in animal models. Therefore, the subject matter of the present invention is to provide molecules useful in the treatment of anxiety-related disorders or other clinical manifestations requiring the use of anxiolytics.

In another aspect, the isolation, structural characterization and evaluation of the pharmacological activity of the molecules of the present invention enable the development of standardized pharmaceutical compositions intended for the treatment of anxiety disorders. Therefore, another subject matter of the present invention is to provide pharmaceutical compositions comprising anxiolytic molecules.

In yet another aspect, the isolation and/or synthesis of the molecules of the present invention provide facilitated processes for producing pharmaceutical compositions comprising said molecules. Therefore, an additional subject matter of the present invention is to provide processes for producing pharmaceutical compositions.

### DESCRIPTION OF THE FIGURE

FIG. 1 displays a general illustrative scheme of the stages of extraction and fractioning of the hydroalcoholic crude extract of E. mulungu and the isolation of the alkaloids erythrartine, erythravine and 11-OH-erythravine using the hydroalcoholic crude extract of E. mulungu.

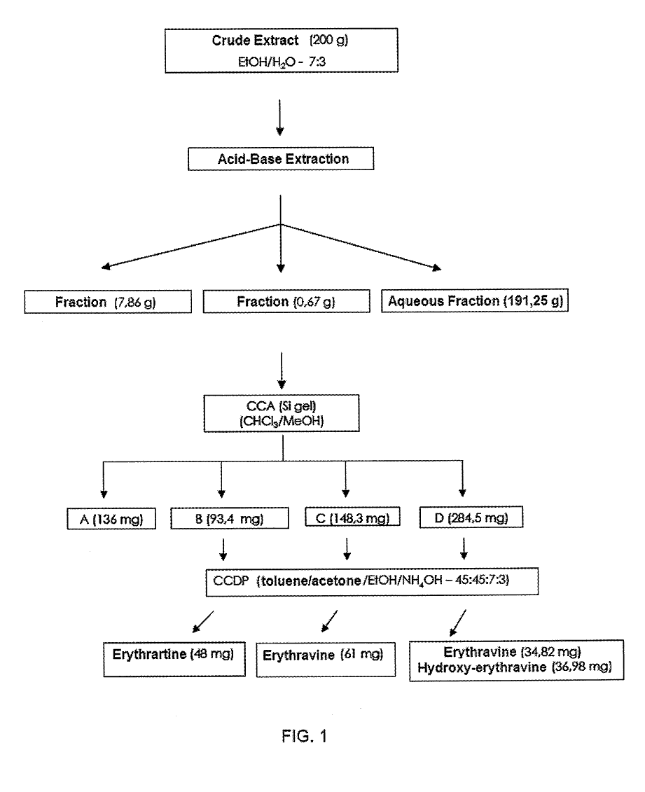

### DETAILED DESCRIPTION OF THE INVENTION

For the purposes of this invention, “pharmaceutical compositions” shall mean all and any composition containing an active principle, having prophylactic, palliative and/or curatives purposes, acting to maintain and/or restore the homeostasis, and may be administered topically, parenterally, enterally and/or intrathecally.

The pharmaceutical compositions referred to in this invention belong to the class of erythrina byproducts and include 11-OH-erythravine, pharmaceutically acceptable isotherals, salts, byproducts and/or solvates thereof, optionally comprising erythrartine and/or erythravine isolates of Erythrina mulungu or chemical synthetics.

The therapeutic applicability of the compounds of the present invention was carried out in various stages. The experiments performed and the respective results are presented below merely as examples, and do not limit the scope of the attached claims.

### Example 1

### Extract Preparation and Fractioning Using Vegetable Material

Flowers were collected from adult trees during the winter season in the county of Rifaina (SP). The fresh vegetable material (6 kg) was submitted to extract by process of maceration with ethanol/water (EtOH/H₂O) (7:3) for 7 days. Next the extract was filtered and concentrated with the assistance of a rota-evaporator, resulting in 292 g of dry hydroalcoholic extract. Then, biomonitored fractioning and isolation of the chemical constituents was carried out. Accordingly, acid-base extraction was performed with the intent of optimizing the separation of the erythrina alkaloids. To achieve this, the dry hydroalcoholic extract (120 g) was dissolved in an aqueous solution of acetic acid (10%) and submitted to liquid/liquid with chloroform extraction (CHCl₃). The chloroform phase was separated from the aqueous phase and the solvent evaporated, resulting in fraction 1 (7.83 g). Next, the aqueous phase was alkalinized with ammonium hydroxide (NH₄OH) in a volume sufficient to attain a pH of between 9-10 and was again submitted to extraction with CHCl₃. The chloroform phase was separated and the solvent evaporated, resulting in fraction 2 (F2) (670 mg).

### Example 2

### Chromatography, Instrumentation and Spectrometry

Degree solvents were used “for analysis”. For analytical thin-layer chromatography (CCD) of silica, CHCl₃/methanol (MeOH) (9:1) was used as the solvents system. The Dragendorf test was positive for alkaloids in F2, which was submitted to open-column chromatography (CCA) (5 cm in diameter and 15 cm high). For the CCA (0.035-0.070 mm, φ 6 ηm) was used as silica stationary phase and CHCl₃/MeOH (10:0-8:2) as mobile phase. For separation, 670 mg of F2 was used, and 101 fractions of approximately 20 ml were collected. After having been submitted to analytical CCD, in mobile phase of CHCl₃/MeOH (7:1) and revealed by the Dragendorf assay, the 101 fractions were grouped in fraction A (FA—136.2 mg) (1-27), fraction B (FB—93.4 mg) (28-50), fraction C (FC—148.3 mg) (51-69), fraction D (FD—284.5 mg)(70-101). To isolate and purify the alkaloids, preparative thin-layer chromatography (CCDP) was used, employing fluoresceine for the silica stationary phase (Merck) and toluene, acetone, ethanol and NH₄OH (45:45:7:3) for the mobile phase. For the spectrometric analyses of the substances isolated from CCDP, a nuclear magnetic resonance (NMR) spectrometer Varian Unit was used, operating at 500 MHz. Deuterated chloroform (CDCl₃) was used as solvent. To determine the chemical structures of the isolated alkaloids, NMR spectrometries of ¹H and ¹³C were used, as well as HMQC, HMBC and COSY bidimensional spectrometry. The results were compared to the information currently available in literature on erythrina alkaloids. Alkaloid 1 was isolated by means of CCDP carried out with FB. Alkaloid 2 was isolated both from FC, as from FD. Using FD, it was also possible to isolate alkaloid 3. The NMR spectrum of ¹H and ¹³C in CDCl₃ for substances 1, 2 and 3 (table 1) showed the presence of signs characteristic of the skeleton of erythrina alkaloids.

TABLE 1
Chemical displacement measurements (δ) and coupling (J) of NMR (500 MHz)
of ¹H and ¹³C (in CD₃Cl) of erythrartine, erythravine and 11-OH-erythravine.

| | erythrartine | | | erythravine | | | 11-OH-erythravine | | |
|---|---|---|---|---|---|---|---|---|---|
| | δ_{H} (J in Hz) | | δ_{C} | δ_{H} (J in Hz) | | δ_{C} | δ_{H} (J in Hz) | | δ_{C} |
| 1 | 5.94 | (d; 10, 5) | 131.52 | 5.9 | (d 10, 0) | 134.18 | 6.01 | (d; 15, 5) | 135 |
| 2 | 6.52 | (dd; 10, 5; 2, 5) | 125.53 | 6.4 | (dd; 10, 0; 1, 5) | 124.96 | 6.56 | (dd) | 124.5 |
| 3 | 3.98 | m | 75.98 | 4.37 | m | 67.73 | 4.5 | m | 67.27 |
| 4 | 1.75 | t | 40.46 | 1.91 | t | 45.33 | 1.91 | t | 43.9 |
| | 2.3 | (dd; 11, 5; 3, 5) | | 2.5 | (dd; 11, 0; 5, 0) | | 2.57 | dd | |
| 5 | — | | 66.30 | — | | 67.11 | — | | |
| 6 | — | | 142.00 | — | | 141.88 | — | | 141.32 |
| 7 | 5.67 | s | 123.50 | 5.66 | s | 122.81 | 5.76 | s | 124.45 |
| 8 | 3.81 | (d; 3, 0) | 58.71 | 3.58 | d | 56.45 | 3.99 | d | 58.78 |
| | 3.88 | (dd) | | 3.7 | d | | 3.93 | d | |
| 10 | 3.54 | (dd; 14, 0; 3, 5) | 50.92 | 3.05 | (dd; 6, 0; 4, 5) | 43.40 | 3.10 | dd | 50.83 |
| | 3.07 | (dd; 14, 0; 4.5) | | 3.50-3.53 | m | | 3.59 | dd | |
| 11 | 4.64 | t | 64.55 | 2.9 | (dd, 10, 0; 6, 5) | 23.76 | 4.74 | t | 63.69 |
| | | | | 2.6-2.7 | m | | | | |
| 12 | — | | 128.32 | — | | 126.01 | — | | ? |
| 13 | — | | 129.68 | — | | 130.61 | — | | ? |
| 14 | 6.91 | s | 108.68 | 6.64 | s | 111.52 | 6.71 | s | 108.36 |
| 15 | — | | 148.28 | — | | 147.19 | — | | 141.32 |
| 16 | — | | 148.52 | — | | 147.77 | — | | 148.76 |
| 17 | 6.77 | s | 110.33 | 6.83 | s | 109.16 | 6.93 | s | 110.36 |

As shown in table 1, it is possible to identify signs of two singlets for the aromatic protons relating to hydrogens H-14 and H-17 and two singlets attributed to methoxyl hydrogens in the position of carbons C-15 and C-16. The presence of three signs of olefinic protons (broad singlet (sl), H-7; broad doublet (dl), H-1; two-doublet (dd), H-2), may be attributed to the dienic system hydrogens of the erythrina skeleton. Although previous works (Sarragioto et al., 1982) had reported resonances of C-1 and C-2 in δ 125.3 and δ 131.2, respectively, in the development of the present invention, the correlation between the chemical displacements of the HMQC bidimensional spectra demonstrated that these resonances occur at δ 131.5 and δ 125.5, respectively.

### 11-hydroxy-erythravine (11-OH-erythravine)

In the NMR spectrum of ¹H for 11-OH-erythravine (table 2), similarly to erythravine, there was no sign of methoxyl hydrogens in the position C-3, as for erythrartine, only a multiplet at δ 4.5 relating to a oxygenated substitute, attributed to the position C-3. In the same way as observed for erythrartine, the spectra of NMR ¹H and of ¹³C revealed chemical displacements at δ 4.74 (t) and δ 63.69, respectively, attributed to the presence of a hydroxyl in C-11. These results are being reported for the first time and, therefore, the substance 3 was recognized as being a new erythrina alkaloid, and was named 11-hidroxi-erythravine (11-OH-erythravine). The chemical structure of the alkaloid 11-OH-erythravine, isolated from the crude extract of E. mulungu flowers is presented below.

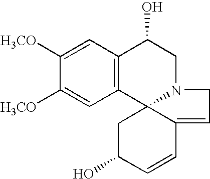

### Example 3

### Pharmacological Evaluation

Swiss mice weighing 25-35 g from the central animal laboratory of the Sao Paulo State University (UNESP/Araraquara) were used. The animals were housed in groups of 10-12 animals, in polypropylene cages with wood shavings on the floor, with food and water available ad lithium]. The animal laboratory was maintained under constant temperature 22±1° C., the lighting was controlled in 12-hour cycles from 7:00 am to 7:00 pm and the humidity was kept at between 50-60%. The pharmacological evaluation was performed with extract, standard drug and vehicle. Accordingly, lyophilized hydroalcoholic extract (50, 100, 200 and 400 mg/kg) was used, in addition to F2 (3, 6, 10, 17 and 30 mg/kg) and the alkaloids erythrartine, erythravine and 11-OH-erythravine (3 and 10 mg/kg), administered via oral intake by gavage. The standard drug used was Diazepam (DZP) in a dose of 2 mg/kg (via intraperitoneal, i.p). All solutions were prepared on the day of the experiment with sodium chlorate 0.9% and sonicated for 15 minutes and the diazepam in a solution of sodium chlorate 0.9% and Tween-80. The experiments were carried out between 11:00 am and 5:00 pm, and the experiment apparatus and procedures are described ahead. The elevated T maze is made with transparent glass walls and wooden floor and consists of a closed arm (30×5×15 cm) perpendicularly linked to two open arms (30×5×0.25 cm), raised at 38.5 cm above the floor by a wooden support. In this test, five consecutive measures of inhibitory avoidance were performed (basal latency, avoidances 1, 2, 3 and 4) and one measure of escape from the open arms, with intervals of 30 seconds between each attempt. In the avoidance measures, the animals were placed in the distal section of the closed arm and the exit latency of this arm, on all four paws, towards the open arm was timed. In the escape measure, the animals were placed in the extremity of the right-hand open arm and the departure time from this arm was measured. The animals' maximum length of stay in the arms of the maze during these measures was 300 seconds. The apparatus was cleaned with ethanol 20% after testing each animal. In order to avoid false positives or negatives, immediately after testing in the elevated T maze, the animals were submitted to the locomotive activity test in the arena. The apparatus consists of a white polypropylene box with a rectangular base (40×48 cm), surrounded by 30 cm high walls. The floor is subdivided into 30 squares (8×8 cm). In this test, the animals were placed in the center of the box and their activity was video recorded for five minutes, for subsequent analysis of the number of crossings of the quadrant areas and number of stretch-attend postures (WALSH and CUMMINS, 1976).

All the results from the animal models were initially submitted to the Levene homogeneity test. The heterogeneous results were converted into a logarithmic scale and later analyzed statistically. The results obtained from the elevated T maze were submitted to a two-way analysis of variance (ANOVA), with treatment being an independent factor and attempts being a dependent factor. When the effect of treatment proved to be significant, the data were analyzed using the one-way ANOVA followed by the Duncan post hoc test. The results obtained with the arena were submitted to a one-way ANOVA followed by the Duncan post hoc test. Amounts p≦0.05 were considered to be significant results.

### Elevated T Maze Test

As can be seen in table 2, the 11-OH-erythravine impaired the performance of the animals in the inhibitory avoidance task in the elevated T maze model. The two-way ANOVA revealed a significant difference in treatment (F(3, 33)=8.30; p<0.001), of the attempts (F(4, 132)=14.75; p<0.0001) and the interaction between treatment and attempts (F(12, 132)=2.42; p<0.01). The one-way ANOVA showed significant differences between the treatment groups in E1(F(3,33)=4.47; p<0.01), E2 (F(3,33)=5.29; p<0.01), E3 (F(3,33)=5.29; p<0.01) and E4(F(3,33)=10.29; p<0.0001), but not in the basal latency (F(3,33)=0.51; p<0.67). Table 2 shows the difference between the groups compared with the control group, according to the Duncan post hoc test.

In relation to the measure of escape from the open arms, the one-way ANOVA showed that there was no difference between the treatment groups when compared to the control group (F(3, 33)=0.71; p<0.54).

TABLE 2
Effect (average + EPM) of the acute treatment with the 11-OH-erythravine
in mice submitted to the elevated T maze test

| | Inhibitory avoidances | | | | | |
|---|---|---|---|---|---|---|
| Treatments | LB | E1 | E2 | E3 | E4 | Escape |
| SALINA | 32.6 ± 5.3 | 107.7 ± 37.9 | 142.6 ± 41.7 | 165.0 ± 37.7 | 232.8 ± 35.5 | 21.8 ± 3.1 |
| DZP 2 mg/kg | 24.6 ± 4.1 | 15.3 ± 3.8* | 12.25 ± 1.6* | 22.3 ± 7.7* | 24.2 ± 7.3* | 19.0 ± 3.2 |
| 11-OH 3 mg/kg | 30.7 ± 5.8 | 41.8 ± 14.2 | 96.0 ± 34.4 | 132.3 ± 39.7 | 194.4 ± 39.5 | 21.5 ± 3.7 |
| 11-OH 10 mg/kg | 22.6 ± 3.5 | 30.0 ± 6.1* | 54.0 ± 21.1 | 66.6 ± 30.3* | 94.8 ± 31.7* | 26.8 ± 4.8 |

*p < 0.05.

### Locomotive Activity—Arena

The one-way ANOVA showed that none of the doses of 11-OH-erythravine used altered the locomotive activity, both in terms of number of crossings of the arena quadrants (F(3, 33)=0.76; p<0.51), as for the number of stretch-attend postures (F(3, 33)=1.20; p<0.32). Table 3 shows the results obtained with the locomotive activity test.

TABLE 3
Effect (average + EPM) of the acute treatment with the 11-OH-
erythravine on the locomotive activity of the mice in the arena.

| Treatments | Crossings | Stretch-attend postures |
|---|---|---|
| SALINA | 153.2 ± 22.66 | 25.2 ± 3.76 |
| DZP 2 mg/kg | 154.6 ± 19.48 | 19.13 ± 2.03 |
| 11-OH 3 mg/kg | 158.5 ± 7.12 | 27.7 ± 3.20 |
| 11-OH 10 mg/kg | 126.5 ± 12.71 | 22.4 ± 3.50 |

1. Hydroalcoholic extract of Erythrina mulungu characterized by comprising a substance having the general formula: 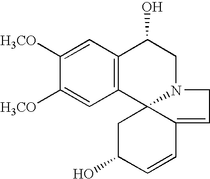
or pharmaceutically acceptable isosters, salts, and/or solvates thereof, associated to dysfunctions of the cholinergic and/or serotonergic system.

2. Hydroalcoholic extract, according to claim 1, characterized by disorders associated to dysfunctions of the cholinergic and/or serotonergic system is anxiety.

3. Pharmaceutical composition containing an hydroalcoholic extract of Erythrina mulungu characterized by comprising a pharmaceutically acceptable vehicle and at least one active substance of the general formula: 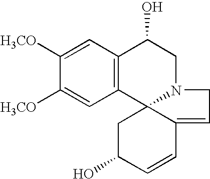
or pharmaceutically acceptable isosters, salts, and/or solvates thereof;
for the treatment of disorders associated to dysfunctions of the cholinergic and/or serotonergic system.

4. Pharmaceutical composition according to claim 3, characterized by disorders associated to dysfunctions of the cholinergic and/or serotonergic system is anxiety.

5. Pharmaceutical composition according to claim 3, characterized by also comprising erythrartine and/or erythravine.

6. Process for producing medicine for the treatment of disorders associated to dysfunctions of the cholinergic and/or serotonergic system characterized by comprising the steps of:
preparing a pharmaceutically acceptable vehicle; and
incorporating into said vehicle at least one active substance of the general formula: 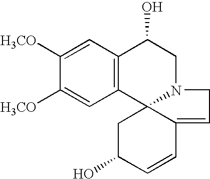
or pharmaceutically acceptable isosters, salts, and/or solvates thereof.

7. Process according to claim 6, characterized by optionally adding the active substance erythrartine and/or erythravine into said vehicle.

8. Process according to claim 6, characterized by the fact that said active substance is obtained from the hydroalcoholic extraction of plants of the Erythrina genus.

9. Process according to claim 8, characterized by the fact that said active substance is obtained from the hydroalcoholic extraction of Erythrina mulungu plants species.

10. Pharmaceutical composition according to claim 4, characterized by also comprising erythrartine and/or erythravine.
